(19) United States
(12) Patent Application Publication
(10) Pub. No.: US 2007/0219175 A1
(43) Pub. Date: Sep. 20, 2007

(54) Controlled Release Pharmaceutical Composition Comprising An Acid-Insoluble And A Bioadhesive Polymer

(75) Inventors: Rajesh Jain, New Delhi (IN); Kour Chand Jindal, New Delhi (IN); Sukhjeet Singh, New Delhi (IN)

Correspondence Address: LADAS & PARRY, 26 WEST 61ST STREET, NEW YORK, NY, 10023, US

(73) Assignee: PANACEA BIOTEC LTD., New Delhi (IN)

(21) Appl. No.: 10/551,058

(22) PCT Filed: Jan. 5, 2005
(86) PCT No.: PCT/IN05/00005

§ 371 Date: Feb. 8, 2007

(30) Foreign Application Priority Data
Jan. 6, 2004 (IN) 22/DEL/2004
Jan. 6, 2004 (IN) 27/DEL/2004

(51) Int. Cl. A61K 31/43

(52) U.S. Cl. 514/192

(57) ABSTRACT

Rapidly disintegrating oral controlled release pharmaceutical compositions and process for preparation of such compositions are provided. The compositions preferably comprise antibiotic(s) as active ingredient, more preferably amoxicillin either alone or in combination with other antibiotic(s). The controlled release compositions comprise at least one active ingredient, and a polymer system comprising of at least two polymers which retard the release of the active ingredient in the stomach while providing rapid release of the said active ingredient in the alkaline contents of small intestine, optionally with other pharmaceutically acceptable excipients. The compositions provide therapeutically effective levels of the active ingredient for extended periods of time, and possess bioadhesive properties.

### FIELD OF THE INVENTION

The present invention relates to controlled release pharmaceutical compositions and process for preparation of such compositions, preferably comprising antibiotic(s) as active ingredient, more preferably Amoxicillin either alone or in combination with other antibiotic(s). The controlled release compositions are of disintegrating type, and additionally possess mucoadhesive properties.

The controlled release composition is useful in providing therapeutically effective levels of the said active ingredient for extended periods of time. Moreover the said composition is expected not to compromise the bioavailability of the active ingredient under fed or fasted conditions.

### BACKGROUND OF THE INVENTION

Amoxicillin is a beta-lactam widely used as a broad-spectrum antibiotic for treatment of a variety of common bacterial infections. Amoxicillin has known susceptibility to inhibition by beta-lactamases produced by resistant organisms. Amoxicillin is available in a variety of formulations, for instance as capsules, tablets, dry powders for reconstitution, chewable tablets, dispersible tablets etc. Amoxicillin is available as tablets of different strengths such as 250 mg, 500 mg, 875 mg etc. The standard adult dose is 250 mg to 500 mg three times a day (tid). In addition, the 875 mg tablet is intended for dosing twice daily (bid) instead of 500 mg tid. A high dose of 3 g, bid is recommended for treatment of recurrent purulent infection of respiratory tract. Use of 1 g Amoxicillin is recommended as one arm of combination therapy, for eradication of helicobacter pylori in peptic ulcer disease.

In the past, attempts have been made to develop modified release/controlled release formulations of Amoxicillin. Such modified/controlled release tablets may provide better patient compliance since they need to be administered twice daily as compared to the 500 mg dose given tid.

European patent number EP1044680 discloses bilayered tablets comprising of an immediate release dose of a part of Amoxicillin and potassium clavulanate and a controlled release dose of a second part of Amoxicillin. The controlled release layer is a hydrophilic matrix. The above said composition suffers from the drawback that it requires excess quantities of excipients for preparing bilayered tablets. This combined with the high dose of Amoxicillin results in a product which is too bulky and difficult to administer.

U.S. Pat. No. 5,690,959 discloses a composition prepared using hydrophobic material manufactured by a process of thermal infusion. Amoxicillin, being temperature sensitive, may undergo degradation if subjected to high temperatures for longer periods of time.

U.S. Pat. No. 6,399,086 discloses a pharmaceutical composition of Amoxicillin wherein 50% of the drug is released within 3-4 hours. The said composition is based on hydrophilic erodible polymers.

U.S. Pat. No. 6,368,635 discloses a solid matrix composition which is solid at ambient temperature, which comprises a viscogenic agent, such as an acrylic acid polymer, capable of developing viscosity on contact with water, as dispersed at least in the neighborhood of the surface layer of a matrix particle containing a polyglycerol fatty acid ester or a lipid and an active ingredient. The matrix may be such that a matrix particle containing a polyglycerol fatty acid ester or a lipid and an active ingredient has been coated with a coating composition containing at least one viscogenic agent. Such composition can adhere to the digestive tract and remain there for a prolonged period of time, thereby increasing the bioavailability of the active ingredient. Such gastric mucosa-adherent particles have unpredictable residence time in the stomach and are highly influenced by the gastric contents. Bioavailability of active agents from such compositions are highly variable.

European patent no. EP0526862 discloses a pharmaceutical composition of Amoxicillin with prolonged residence due to high density of the composition. The said composition suffers from the drawback that non-uniform release of active ingredient results due to variable passage of tablet into intestine by virtue of density itself resulting in significant bioavailability loss.

Hilton and Deasy, [J. Pharm. Sci. 82(7):737-743 (1993)] describe a controlled-release tablet of Amoxicillin trihydrate based on the enteric polymer hydroxypropylmethyl cellulose acetate succinate. This polymer suppressed the release of the drug in the presence of gastric pH but could enhance its release in the small intestine. Therefore, such a formulation cannot give the desired burst effect outlined in the present invention. Single dose studies with a panel of fasting subjects showed that the tablets had a relative bioavailability of only 64.4%, probably because of the poorer absorption of Amoxicillin from the distal jejunum and ileum than from the duodenum and proximal jejunum. Other pharmacokinetic parameters confirmed a lack of therapeutic advantage of these factors over an equivalent dose of conventional capsule.

Hilton and Deasy [Int. J. Pharm. 86(1):79-88 (1992)] also describe a floating tablet of Amoxicillin trihydrate. A bilayer tablet was initially formed in which the controlled-release drug layer consisted of Amoxicillin and hydroxypropyl cellulose. This layer was bonded to a gas generating layer. However, when the two layers were joined together, the composite tablet failed to float and prematurely split along the joining of the two layers. Consequently, it was decided to abandon this approach in favor of a single-layer floating tablet. This tablet remained buoyant for 6 hours and had satisfactory in vitro sustained release. However, compared with conventional capsules in fasting humans at 500 mg equivalent dose of Amoxicillin, the relative bioavailability of the tablets were 80.5% and other pharmacokinetic parameters T(0.1 mug/ml) and T(0.5 mug/ml) corresponding to the length of time for which the serum levels remained greater than or equal to 0.1 mug/ml and 0.5 mug/ml, respectively, indicated lack of improved efficacy.

Uchida et al. [Chem. Pharm. Bull. 37(12):3416-3419 (1989)] describe a preparation of Amoxicillin, microencapsulated in ethyl cellulose. These micro-capsules exhibited a sustained-release effect when administered to dogs. However, such effect could be foreseen, since the gastric pH of the dogs which were tested, is considerably higher than human gastric pH (pH of about 6 in beagle dogs, compared to pH of about 2 in humans). The Amoxicillin is much less soluble at pH 6 than at pH 2. One would expect to obtain a very quick release of the drug from the same microcapsules if administered to humans. Hence, such combination would not provide a controlled release of Amoxicillin Arancibia et al. [Int. J. Clin. Pharmacol. Ther. Toxicol. 25(2):97-100 (1987)] investigated the pharmacokinetics and bioavailability of Amoxicillin trihydrate. They refer to controlled-release tablets, the composition of which is not described. In any case, no drug was detectable after 8 hours from oral administration and therefore this formulation had no advantage over conventional formulations.

Some of the compositions discussed in the art are prepared using hydrophilic swellable polymers. However, these compositions require the use of excessive quantities of release controlling agents. This combined with high dose of amoxicillin, results in a product, which is too bulky to administer orally. In addition, these products have significant food effects resulting in variable bioavailability. Another approach available in the art involves the use of bioadhesive polymers. Such products are highly variable since bioadhesiveness is a property, which is significantly dependent of the gastric contents. Presence of food in the stomach reduces the bioadhesive property resulting in reduced bioavailability. A third approach discussed in the art uses enteric polymers. Since Amoxicillin is predominantly absorbed from proximal part of small intestine, enteric release of the drug results in loss of bioavailability. Hence there still exists a need for developing controlled release compositions of amoxicillin, either alone or in combination with other antibiotic(s) devoid of limitations discussed above.

### SUMMARY OF THE INVENTION

It is an objective of the present invention to provide rapidly disintegrating oral controlled release pharmaceutical composition comprising at least one active ingredient, and a polymer system comprising of at least two polymers wherein one is an acid insoluble polymer and the other is a bioadhesive polymer, which retard the release of the active ingredient in the stomach while providing rapid release of the said active ingredient in the pH above 5.5, optionally with other pharmaceutically acceptable excipients.

It is an objective of the present invention to provide rapidly disintegrating oral controlled release pharmaceutical composition comprising at least one active ingredient preferably antibiotic, more preferably amoxicillin or its pharmaceutically acceptable salts, hydrates, polymorphs, esters, and derivatives thereof.

It is a further objective of the present invention to provide controlled release composition comprising an antibiotic as an active ingredient in combination with at least one other antibiotic.

It is yet another objective of the present invention to provide process for the preparation of such composition which comprises of the following steps:

i) mixing of active ingredient(s) and polymer(s),

ii) optionally adding one or more other pharmaceutically acceptable excipients, and

iii) formulation of the mixture into a suitable dosage form.

### DETAILED DESCRIPTION OF THE INVENTION

The present invention relates to rapidly disintegrating oral controlled release pharmaceutical composition comprising at least one active ingredient or its pharmaceutically acceptable salts, hydrates, polymorphs, esters, and derivatives thereof; and a polymer system, optionally with other pharmaceutically acceptable excipients. The polymer system comprises of at least two polymers, wherein one is an acid insoluble polymer and the other is a bioadhesive polymer. The polymer system retards the release of the active ingredient in the stomach while providing rapid release of the said active ingredient in the pH above 5.5.

In an embodiment, the present invention describes controlled release mucoadhesive, disintegrating type formulation of Amoxicillin, preferably in its trihydrate form. The said composition disintegrates into particles, which have increased residence time in the stomach thus maintaining concentrations above effective levels for extended periods of time. The controlled release formulation provides better patient compliance since they need to be administered twice daily as compared to 500 mg dose given tid.

The present invention also relates to controlled release compositions of preferably an antibiotic, more preferably amoxicillin trihydrate, either alone or in combination with other antibiotic(s) for maintaining concentrations above effective levels, for extended periods of time. The release mechanism involves predominantly diffusion and the product is preferably in the form of a rapidly disintegrating tablet.

The controlled release compositions prepared according to the present invention provides for rapidly disintegrating tablet where the granules behave as controlled release particles. These particles have a unique polymer combination to retard the release in the stomach while providing rapid dissolution in the alkaline contents of small intestine. In addition, the controlled release compositions have bioadhesive properties.

In an embodiment of the present invention, the controlled release composition comprises an antibiotic as an active ingredient in combination with at least one other antibiotic. The antibiotics are selected from but not limited to the group comprising amoxicillin, ampicillin, cloxacillin, clavulanic acid, cephalosporins, and the like.

In an embodiment, the active ingredient of the present pharmaceutical composition is cephalexin, or its pharmaceutically acceptable salts, hydrates, polymorphs, esters, and derivatives thereof.

The polymer system of the present invention comprises of polymer system comprises of polymers selected from a group comprising polyvinyl pyrrolidone, polyvinyl acetate, methacrylic acid polymers, acrylic acid polymers, hydroxypropyl methylcellulose phthalate, hydroxypropyl methylcellulose acetate succinate, cellulose acetate phthalate, cellulose acetate butyrate, cellulose acetate propionate, and alginates, cellulose derivative, polyethylene oxide, chitosans, and polycarbophil, or mixtures thereof. Preferably the polymer system comprises methacrylic acid polymer and polycarbophil.

The acid insoluble polymer of the present invention is selected form but not limited to a group comprising methacrylic acid polymers, acrylic acid polymers, hydroxypropyl methylcellulose phthalate, hydroxypropyl methylcellulose acetate succinate, cellulose acetate phthalate, cellulose acetate butyrate, cellulose acetate propionate, alginates, and the like; or mixtures thereof and the other is a bioadhesive polymer is selected form but not limited to a group comprising polycarbophil such as Noveon® AA1 (B. F. Goodrich Specialty Polymers), and chitosans, or mixtures thereof. Polycarbophil is a polyacrylic acid that is cross-linked with divinyl glycol.

The methacrylic acid polymer is selected from a group comprising but not limited to Eudragit® ((Degussa) such as Eudragit® L-100, Ammonio Methacrylate Copolymer type USP (Eudragit® RL), Ammonio Methacrylate Copolymer type B USP (Eudragit® RS), Eudragit® RSPO, Eudragit® RLPO, and Eudragit® RS30D.

In a preferred embodiment of the present invention, the rapidly disintegrating oral controlled release pharmaceutical composition comprises amoxicillin trihydrate; and a polymer system comprising methacrylic acid polymer and polycarbophil, optionally with other pharmaceutically acceptable excipients.

In an embodiment of the present invention, the ratio of methacrylic acid polymer and polycarbophil is 20:1 to 1:20 by weight of the composition. Preferably the ratio of methacrylic acid polymer and polycarbophil is 10:1 to 1:10 by weight of the composition.

In another preferred embodiment of the present invention, the composition additionally comprises a cellulose derivative, selected from but not limited to a group comprising alkyl cellulose such as ethyl cellulose, methyl cellulose, and the like; carboxyalkyl cellulose such as carboxyethyl cellulose, carboxymethyl cellulose, carboxypropyl cellulose, and the like, and hydroxyalkyl cellulose such as hydroxyethyl cellulose, hydroxymethyl cellulose, hydroxypropyl cellulose, and the like, and hydroxypropyl alkyl cellulose such as hydroxypropyl methyl cellulose, and the like. Preferably, the cellulose derivative is alkyl cellulose such as ethylcellulose or propylcellulose.

The pharmaceutically acceptable excipients of the present invention are selected from the group comprising diluents disintegrants, binders, fillers, bulking agent, coating agents, plasticizers, organic solvents, colourants, stabilizers, preservatives, lubricants, glidants, chelating agents, and the like known to the art.

In an embodiment of the present invention is provided a process for preparation of composition as herein described which comprises of the following steps:

i) mixing of active ingredient(s) and polymer(s),

ii) optionally adding one or more other pharmaceutically acceptable excipients, and

iii) formulation of the mixture into a suitable dosage form.

In an embodiment, the composition of the present invention is in the form of tablets. The tablets can be prepared by either direct compression, dry compression (slugging), or by granulation.

The granulation technique is either aqueous or non-aqueous. Preferably, the tablets of the present invention are prepared by non-aqueous granulation technique. The non-aqueous solvent used is selected from a group comprising ethanol or isopropyl alcohol.

In yet another embodiment, the controlled release formulations prepared according to the present invention disintegrates into particles, which adhere to mucosa of the stomach. These particles provide for controlled release of Amoxicillin till the time they are retained in the stomach. Passage of these granules into the small intestine results in dissolution of release controlling polymers, thus liberating any residual drug entrapped in the particles. This unique combination of polymers provides for a controlled release formulation which does not result in significant loss of bioavailability. Such a formulation does not involve the use of swellable polymers, hydrophobic waxy materials. Such a product may be prepared using polymers like polyvinyl pyrrolidone, polyvinyl acetate, methacrylic acid polymers, acrylic acid polymers; and the like either alone or in combination thereof.

The controlled release composition of the present invention may be formulated as oral dosage forms such as tablets, capsules and the like.

The examples given below serve to illustrate embodiments of the present invention. However they do not intend to limit the scope of present invention.

### EXAMPLES

### Example 1

A. Core Granules

mg/tablet

Ingredients

| | | |
|---|---|---|
| i) | Amoxicillin trihydrate | 860 |
| | (equivalent to 750 mg of Amoxicillin) | |
| ii) | Eudragit ® L-100 | 180 |
| iii) | Polycarbophil | 70 |
| iv) | Eudragit ® L-100 (Binder) | 20 |
| v) | Isopropyl Alcohol | Lost in processing |
| vi) | Dichloromethane | Lost in processing |

Procedure:

| | |
|---|---|
| 1. | Mix (i), (ii) and (iii). |
| 2. | Dissolve (iv) in 1:2 mixture of (v) and (vi). |
| 3. | Granulate the blend of step 1 with solution of step 2. |
| 4. | Pass the wet mass through sieve of mesh size 20 and dry. |
| 5. | Pass the dried granule through sieve of mesh size 30. |

B. Coating of the Granules in FBC (Fluid Bed Coater)

% w/w

Ingredients

| | | |
|---|---|---|
| i) | Eudragit ® L-100 | 12.5 |
| ii) | Polycarbophil | 0.625 |
| iii) | Triethyl citrate | 2.5 |
| iv) | Isopropyl alcohol | q.s. |
| v) | Dichloromethane | q.s. |
| vi) | Colour lake of Poncaou 4R | 0.1 |

Procedure:

| | |
|---|---|
| 1. | Mix (i) and (ii) |
| 2. | Pass (vi) through sieve of mesh no. 120. |
| 3. | Disperse the bulk of step 1 and 2 in 1:2 mixture of (iv) and (v). |
| 4. | Add (iii) to the bulk of step 3 and stir. |
| 5. | Coat the granules of part A in FBC with the solution B. |

C. Compression

mg/tablet

Ingredient

| | | |
|---|---|---|
| i) | Amoxicillin granules (coated in B) | 1399.7 |
| ii) | Microcrystalline cellulose | 100.0 |
| iii) | Croscarmellose sodium | 50.0 |
| iv) | Talc | 10.0 |
| v) | Magnesium stearate | 10.0 |

Procedure:

| | |
|---|---|
| 1. | Mix (ii), (iii), (iv) and (v) |
| 2. | Pass the mixture of step 1 through mesh no. 40 and blend with (i) |
| 3. | Compress the blended granules into tablets. |

### Example 2

A. Core Granules

mg/tablet

Ingredients

| | | |
|---|---|---|
| i) | Amoxicillin trihydrate | 860 |
| | (equivalent to 750 mg of Amoxicillin) | |
| ii) | Eudragit ® L-100 | 150 |
| iii) | Polycarbophil | 60 |
| iv) | Eudragit ® L-100 (Binder) | 20 |
| v) | Isopropyl alcohol | Lost in processing |
| vi) | Dichloromethane | Lost in processing |

Procedure:

| | |
|---|---|
| 1. | Mix (i), (ii) and (iii). |
| 2. | Dissolve (iv) in (v). |
| 3. | Granulate the mass of step 1 with solution of step 2. |
| 4. | Pass the wet mass through sieve of mesh size 20 and dry. |
| 5. | Pass the dried granule through sieve of mesh size 30. |

B. Coating

% w/w

Ingredient

| | | |
|---|---|---|
| i) | Eudragit ® L-100 | 20.0 |
| ii) | Polycarbophil | 1.0 |
| iii) | Triethyl citrate | 2.0 |
| iv) | Isopropyl alcohol | q.s. |
| v) | Dichloromethane | q.s. |
| vi) | Colour lake of Poncaou 4R | 0.1 |

Procedure:

| | |
|---|---|
| 1. | Mix (i) and (ii) |
| 2. | Pass (vi) through sieve of mesh no. 120. |
| 3. | Disperse the bulk of step 1 and 2 in 1:2 mixture of (iv) and (v). |
| 4. | Add (iii) to the bulk of step 3 and stir for 45 minutes. |
| 5. | Coat the granules of part A in FBC with the solution B. |

C. Compression

mg/tablet

Ingredient

| | | |
|---|---|---|
| i) | Amoxicillin granules (coated in B) | 1310.0 |
| ii) | Microcrystalline cellulose | 150.0 |
| iii) | Croscarmellose sodium | 20.0 |
| iv) | Talc | 10.0 |
| v) | Magnesium stearate | 10.0 |

Procedure:

| | |
|---|---|
| 1. | Mix (ii), (iii), (iv) and (v) |
| 2. | Pass the mixture of step 1 through mesh no. 40 and blend with (i) |
| 3. | Compress the blended granules into tablets. |

### Example 3

A. Core Granules

mg/tablet

Ingredients

| | | |
|---|---|---|
| i) | Amoxicillin trihydrate | 860.00 |
| | (equivalent to 750 mg of Amoxicillin) | |
| ii) | Eudragit ® L-100 | 180.00 |
| iii) | Polycarbophil | 70.00 |
| iv) | PVPK-30 | 20.00 |
| v) | Purified Water | Lost in processing |

Procedure:

| | |
|---|---|
| 6. | Mix (i), (ii) and (iii) pass through mesh size 30. |
| 7. | Dissolve (iv) in water |
| 8. | Granulate the mass of step 1 with solution of step 2. |
| 9. | Pass the wet mass through sieve of mesh size 20 and dry. |
| 10. | Pass the dried granule through sieve of mesh size 30. |

B. Coating of the Granules in FBC (Fluid Bed Coater)

% w/w

Ingredients

| | | |
|---|---|---|
| i) | Eudragit ® NE 30 D | 12.50 |
| | (Dry polymer weight of 30% w/w dispersion) | |
| ii) | Polycarbophil | 0.625 |
| iii) | Talc | 6.25 |
| iv) | Colour Lake of Ponceau 4R | 0.10 |
| v) | Purified Water | Lost in processing |

Procedure:

| | |
|---|---|
| 6. | Mix (ii), (iii) and (iv) |
| 7. | Pass mass of step 1 through sieve of mesh no. 100. |
| 8. | Disperse the bulk of step 2 in (v) and pass through a Colloid mill. |
| 9. | Add (i) to the bulk of step 3 and stir. |
| 10. | Coat the granules of part A in FBC with solution of step 4. |

C. Compression

mg/tablet

Ingredients

| | | |
|---|---|---|
| i) | Amoxicillin granules (coated in B) | 1350.09 |
| ii) | Microcrystalline cellulose | 100.00 |
| iii) | Croscarmellose sodium | 50.00 |
| iv) | Talc | 10.00 |
| v) | Magnesium stearate | 10.00 |

Procedure:

| | |
|---|---|
| 4. | Mix (ii), (iii), (iv) and (v) |
| 5. | Pass the mixture of step 1 through mesh no. 40 and blend with (i) |
| 6. | Compress the blended granules into tablets. |

### Example 4

A. Core Granules

mg/tablet

Ingredients

| | | |
|---|---|---|
| i) | Amoxicillin trihydrate | 860.00 |
| | (equivalent to 750 mg of Amoxicillin) | |
| ii) | Eudragit ® L-100 | 100.00 |
| iii) | Polycarbophil | 40.00 |
| iv) | Eudragit ® L-30-D55 | 150.00 |
| | (Dry polymer weight of 30% w/w dispersion) | |
| v) | Purified Water | Lost in processing |

Procedure:

| | |
|---|---|
| 1. | Mix (i), (ii) and (iii) and pass through mesh size 30. |
| 2. | Disperse (iv) in water |
| 3. | Granulate the mass of step 1 with dispersion of step 2. |
| 4. | Pass the wet mass through sieve of mesh size 20 and dry. |
| 5. | Pass the dried granule through sieve of mesh size 30. |

B. Coating of the Granules in FBC (Fluid Bed Coater)

% w/w

Ingredients

| | | |
|---|---|---|
| i) | Eudragit ® L-30-D55 | 12.50 |
| | (Dry polymer weight of 30% w/w dispersion) | |
| ii) | Polycarbophil | 0.625 |
| iii) | Talc | 6.25 |
| iv) | Triethyl Citrate | 1.25 |
| v) | Colour Lake of Ponceau 4R | 0.10 |
| vi) | Purified Water | Lost in processing |

Procedure:

| | |
|---|---|
| 1. | Mix (ii), (iii) and (v). |
| 2. | Pass mass of step 1 through sieve of mesh no. 100. |
| 3. | Disperse the bulk of step 2 in (vi) and pass through a Colloid mill. |
| 4. | Add (i) and (iv) to the bulk of step 3 and stir. |
| 5. | Coat the granules of part A in FBC with solution of step 4. |

C. Compression

mg/tablet

Ingredients

| | | |
|---|---|---|
| i) | Amoxicillin granules (coated in B) | 1388.34 |
| ii) | Microcrystalline cellulose | 100.00 |
| iii) | Croscarmellose sodium | 50.00 |
| iv) | Talc | 10.00 |
| v) | Magnesium stearate | 10.00 |

Procedure:

| | |
|---|---|
| 1. | Mix (ii), (iii), (iv) and (v) |
| 2. | Pass the mixture of step 1 through mesh no. 40 and blend with (i) |
| 3. | Compress the blended granules into tablets. |

### Example 5

A. Core Granules

mg/tablet

Ingredients

| | | |
|---|---|---|
| i) | Amoxicillin trihydrate | 860.00 |
| | (equivalent to 750 mg of Amoxicillin) | |
| ii) | Eudragit ® L-100 | 120.00 |
| iii) | Polycarbophil | 40.00 |
| iv) | Eudragit ® L-30-D55 | 80.00 |
| | (Dry polymer weight of 30% w/w dispersion) | |
| v) | Purified Water | Lost in processing |

Procedure:

| | |
|---|---|
| 1. | Mix (i), (ii) and (iii) pass through mesh size 30. |
| 2. | Disperse (iv) in water |
| 3. | Granulate the mass of step 1 with solution of step 2. |
| 4. | Pass the wet mass through sieve of mesh size 20 and dry. |
| 5. | Pass the dried granule through sieve of mesh size 30. |

B. Coating of the Granules in FBC (Fluid Bed Coater)

% w/w

Ingredients

| | | |
|---|---|---|
| i) | Eudragit ® L-30-D55 | 16.00 |
| | (Dry polymer weight of 30% w/w dispersion) | |
| ii) | Polycarbophil | 0.09 |
| iii) | Talc | 8.00 |
| iv) | Triethyl Citrate | 3.20 |
| v) | Colour Lake of Ponceau 4R | 0.10 |
| vi) | Purified Water | Lost in processing |

Procedure:

| | |
|---|---|
| 1. | Mix (ii), (iii) and (v). |
| 2. | Pass bulk of step 1 through sieve of mesh no. 100. |
| 3. | Disperse the bulk of step 2 in (vi) and pass through a Colloid mill. |
| 4. | Add (i) and (iv) to the bulk of step 3 and stir. |
| 5. | Coat the granules of part A in FBC with solution of step 4. |

C. Compression

mg/tablet

Ingredients

| | | |
|---|---|---|
| i) | Amoxicillin granules (coated in B) | 1401.29 |
| ii) | Microcrystalline cellulose | 100.00 |
| iii) | Croscarmellose sodium | 50.00 |
| iv) | Talc. | 10.00 |
| v) | Magnesium stearate | 10.00 |

Procedure:

| | |
|---|---|
| 1. | Mix (ii), (iii), (iv) and (v) |
| 2. | Pass the mixture of step 1 through mesh no. 40 and blend with (i) |
| 3. | Compress the blended granules into tablets. |

### Example 6

A. Core Granules

mg/tablet

Ingredients

| | | |
|---|---|---|
| i) | Amoxicillin trihydrate | 860.00 |
| | (equivalent to 750 mg of Amoxicillin) | |
| ii) | Ethyl Cellulose M 20 | 100.00 |
| iii) | Polycarbophil | 40.00 |
| iv) | Eudragit ® L-30-D55 | 20.00 |
| | (Dry polymer weight of 30% w/w dispersion) | |
| v) | Purified Water | Lost in processing |

Procedure:

| | |
|---|---|
| 1. | Mix (i) and (iii) pass through mesh size 30. |
| 2. | Pass (ii) through sieve of mesh size 100 and blend with mass of |
| | step 1. |
| 3. | Disperse (iv) in Purified Water. |
| 4. | Granulate the mass of step 2 with solution of step 3. |
| 5. | Pass the wet mass through sieve of mesh size 20 and dry. |
| 6. | Pass the dried granule through sieve of mesh size 30. |

B. Coating of the Granules in FBC (Fluid Bed Coater)

% w/w

Ingredients

| | | |
|---|---|---|
| i) | Eudragit ® L-30-D55 | 12.50 |
| | (Dry polymer weight of 30% w/w dispersion) | |
| ii) | Talc | 6.25 |
| iii) | Triethyl Citrate | 3.75 |
| iv) | Colour Lake of Ponceau 4R | 0.10 |
| v) | Purified Water | Lost in processing |

Procedure:

| | |
|---|---|
| 1. | Mix (iii) and (v). |
| 2. | Pass mass of step 1 through sieve of mesh no. 100. |
| 3. | Disperse the bulk of step 2 in (v) and pass through a Colloid mill. |
| 4. | Add (i) and (iii) to the bulk of step 3 and stir. |
| 5. | Coat the granules of part A in FBC with solution of step 4. |

C. Compression

mg/tablet

Ingredients

| | | |
|---|---|---|
| i) | Amoxicillin granules (coated in B) | 1251.34 |
| ii) | Microcrystalline cellulose | 100.00 |
| iii) | Croscarmellose sodium | 50.00 |
| iv) | Talc | 10.00 |
| v) | Magnesium stearate | 10.00 |

Procedure:

| | |
|---|---|
| 1. | Mix (ii), (iii), (iv) and (v) |
| 2. | Pass the mixture of step 1 through mesh no. 40 and blend with (i) |
| 3. | Compress the blended granules into tablets. |

### Example 7

A. Core Granules

mg/tablet

Ingredients

| | | |
|---|---|---|
| i) | Amoxicillin trihydrate | 860.00 |
| | (equivalent to 750 mg of Amoxicillin) | |
| ii) | Ethyl Cellulose | 20.00 |
| iii) | Polycarbophil | 40.00 |
| iv) | Eudragit ® L100 | 50.00 |
| v) | Eudragit ® L-30-D55 | 100.00 |
| | (Dry polymer weight of 30% w/w dispersion) | |
| vi) | Purified Water | Lost in processing |

Procedure:

| | |
|---|---|
| 1. | Mix (i), (iii) and (iv) pass through mesh size 30. |
| 2. | Pass (ii) through sieve of mesh size 100 and blend with mass of |
| | step 1. |
| 3. | Disperse (v) in Purified Water. |
| 4. | Granulate the mass of step 2 with solution of step 3. |
| 5. | Pass the wet mass through sieve of mesh size 20 and dry. |
| 6. | Pass the dried granule through sieve of mesh size 30. |

B. Coating of the Granules in FBC (Fluid Bed Coater)

% w/w

Ingredients

| | | |
|---|---|---|
| i) | Eudragit ® L-30-D55 | 12.50 |
| | (Dry polymer weight of 30% w/w dispersion) | |
| ii) | Polycarbophil | 0.625 |
| iii) | Talc | 6.25 |
| iv) | Triethyl Citrate | 2.50 |
| v) | Colour Lake of Ponceau 4R | 0.10 |
| vi) | Purified Water | Lost in processing |

Procedure:

| | |
|---|---|
| 1. | Mix (ii), (iii) and (v). |
| 2. | Pass mass of step 1 through sieve of mesh no. 100. |
| 3. | Disperse the bulk of step 2 in (vi) and pass through a Colloid mill. |
| 4. | Add (i) and (iv) to the bulk of step 3 and stir. |
| 5. | Coat the granules of part A in FBC with solution of step 4. |

C. Compression

mg/tablet

Ingredients

| | | |
|---|---|---|
| i) | Amoxicillin granules (coated in B) | 1305.13 |
| ii) | Microcrystalline cellulose | 100.00 |
| iii) | Croscarmellose sodium | 50.00 |
| iv) | Talc | 10.00 |
| v) | Magnesium stearate | 10.00 |

Procedure:

| | |
|---|---|
| 1. | Mix (ii), (iii), (iv) and (v) |
| 2. | Pass the mixture of step 1 through mesh no. 4.0 and blend with (i) |
| 3. | Compress the blended granules into tablets. |

### Example 8

A. Core Granules

mg/tablet

Ingredients

| | | |
|---|---|---|
| i) | Amoxicillin trihydrate | 860.00 |
| | (equivalent to 750 mg of Amoxicillin) | |
| ii) | Eudragit ® RSPO | 100.00 |
| iii) | Polycarbophil | 40.00 |
| iv) | Eudragit ® L-30-D55 | 100.00 |
| | (Dry polymer weight of 30% w/w dispersion) | |
| v) | Purified Water | Lost in processing |

Procedure:

| | |
|---|---|
| 1. | Mix (i), (ii) and (iii) pass through mesh size 30. |
| 2. | Disperse (iv) in Purified Water. |
| 3. | Granulate the mass of step 1 with solution of step 2. |
| 4. | Pass the wet mass through sieve of mesh size 20 and dry. |
| 5. | Pass the dried granule through sieve of mesh size 30. |

B. Coating of the Granules in FBC (Fluid Bed Coater)

% w/w

Ingredients

| | | |
|---|---|---|
| i) | Eudragit ® L-100 | 12.50 |
| ii) | Polycarbophil | 0.625 |
| iii) | Triethyl Citrate | 2.50 |
| iv) | Isopropyl Alcohol | Lost in processing |
| v) | Dichloromethane | Lost in processing |
| vi) | Colour Lake of Ponceau 4R | 0.10 |

Procedure:

| | |
|---|---|
| 1. | Mix (i) and (ii) and pass through mesh no. 100. |
| 2. | Pass (vi) through sieve of mesh no. 120. |
| 3. | Disperse the bulk of step 1 and 2 in 1:2 mixture of (iv) and (v) |
| 4. | Add (iii) to the bulk of step 3 and stir. |
| 5. | Coat the granules of part A in FBC with solution of step 4. |

C. Compression

mg/tablet

Ingredients

| | | |
|---|---|---|
| i) | Amoxicillin granules (coated in B) | 1272.97 |
| ii) | Microcrystalline cellulose | 100.00 |
| iii) | Croscarmellose sodium | 50.00 |
| iv) | Talc | 10.00 |
| v) | Magnesium stearate | 10.00 |

Procedure:

| | |
|---|---|
| 1. | Mix (ii), (iii), (iv) and (v) |
| 2. | Pass the mixture of step 1 through mesh no. 40 and blend |
| | with (i) |
| 3. | Compress the blended granules into tablets. |

### Example 9

A. Core Granules

mg/tablet

Ingredients

| | | |
|---|---|---|
| i) | Amoxicillin trihydrate | 860.00 |
| | (equivalent to 750 mg of Amoxicillin) | |
| ii) | Eudragit RLPO | 100.00 |
| iii) | Polycarbophil | 40.00 |
| iv) | Eudragit L-30-D55 | 100.00 |
| | (Dry polymer weight of 30% w/w dispersion) | |
| v) | Purified Water | Lost in processing |

Procedure:

| | |
|---|---|
| 1. | Mix (i), (ii) and (iii) pass through mesh size 30. |
| 2. | Disperse (iv) in Purified Water |
| 3. | Granulate the mass of step 1 with solution of step 2. |
| 4. | Pass the wet mass through sieve of mesh size 20 and dry. |
| 5. | Pass the dried granule through sieve of mesh size 30. |

B. Coating of the Granules in FBC (Fluid Bed Coater)

% w/w

Ingredients

| | | |
|---|---|---|
| i) | Eudragit L-100 | 12.50 |
| ii) | Polycarbophil | 0.625 |
| iii) | Triethyl Citrate | 2.50 |
| iv) | Isopropyl Alcohol | Lost in processing |
| v) | Dichloromethane | Lost in processing |
| vi) | Colour Lake of Ponceau 4R | 0.10 |

Procedure:

| | |
|---|---|
| 1. | Mix (i) and (ii) and pass through mesh no. 100. |
| 2. | Pass (vi) through sieve of mesh no. 120. |
| 3. | Disperse the bulk of step 1 and 2 in 1:2 mixture of (iv) and (v) |
| 4. | Add (iii) to the bulk of step 3 and stir. |
| 5. | Coat the granules of part A in FBC with solution of step 4. |

C. Compression

mg/tablet

Ingredient

| | | |
|---|---|---|
| i) | Amoxicillin granules (coated in B) | 1272.97 |
| ii) | Microcrystalline cellulose | 100.00 |
| iii) | Croscarmellose sodium | 50.00 |
| iv) | Talc | 10.00 |
| v) | Magnesium stearate | 10.00 |

Procedure:

| | |
|---|---|
| 1. | Mix (ii), (iii), (iv) and (v) |
| 2. | Pass the mixture of step 1 through mesh no. 40 and blend |
| | with (i) |
| 3. | Compress the blended granules into tablets. |

### Example 10

A. Core Granules

mg/tablet

Ingredients

| | | |
|---|---|---|
| i) | Amoxicillin trihydrate | 860.00 |
| | (equivalent to 750 mg of Amoxicillin) | |
| ii) | Eudragit RLPO | 100.00 |
| iii) | Polycarbophil | 40.00 |
| iv) | Triethyl Citrate | 20.00 |
| v) | Eudragit L-30-D55 | 100.00 |
| | (Dry polymer weight of 30% w/w dispersion) | |
| vi) | Purified Water | Lost in processing |

Procedure:

| | |
|---|---|
| 1. | Mix (i), (ii) and (iii) pass through mesh size 30. |
| 2. | Disperse (iv) and (v) in water |
| 3. | Granulate the mass of step 1 with solution of step 2. |
| 4. | Pass the wet mass through sieve of mesh size 20 and dry. |
| 5. | Pass the dried granule through sieve of mesh size 30. |

B. Coating of the Granules in FBC (Fluid Bed Coater)

% w/w

Ingredients

| | | |
|---|---|---|
| i) | Eudragit L-100 | 12.50 |
| ii) | Polycarbophil | 0.625 |
| iii) | Triethyl Citrate | 2.50 |
| iv) | Isopropyl Alcohol | Lost in processing |
| v) | Dichloromethane | Lost in processing |
| vi) | Colour Lake of Ponceau 4R | 0.10 |

Procedure:

| | |
|---|---|
| 1. | Mix (i) and (ii) pass through mesh no. 100. |
| 2. | Pass (vi) through sieve of mesh no. 120. |
| 3. | Disperse the bulk of step 1 and 2 in 1:2 mixture of (iv) and (v) |
| 4. | Add (iii) to the bulk of step 3 and stir. |
| 5. | Coat the granules of part A in FBC with solution of step 4. |

C. Compression

mg/tablet

Ingredients

| | | |
|---|---|---|
| i) | Amoxicillin granules (coated in B) | 1296.12 |
| ii) | Microcrystalline cellulose | 100.00 |
| iii) | Croscarmellose sodium | 50.00 |
| iv) | Talc | 10.00 |
| v) | Magnesium stearate | 10.00 |

Procedure:

| | |
|---|---|
| 1. | Mix (ii), (iii), (iv) and (v) |
| 2. | Pass the mixture of step 1 through mesh no. 40 and blend |
| | with (i) |
| 3. | Compress the blended granules into tablets. |

### Example 11

A. Core Granules

mg/tablet

Ingredients

| | | |
|---|---|---|
| i) | Amoxicillin trihydrate | 860.00 |
| | (equivalent to 750 mg of Amoxicillin) | |
| ii) | Eudragit RLPO | 100.00 |
| iii) | Polycarbophil | 40.00 |
| iv) | Triethyl Citrate | 20.00 |
| iv) | Eudragit L-30-D55 | 100.00 |
| | (Dry polymer weight of 30% w/w dispersion) | |
| v) | Purified Water | Lost in processing |

Procedure:

| | |
|---|---|
| 1. | Mix (i), (ii) and (iii) pass through mesh size 30. |
| 2. | Disperse (iv) and (v) in Purified Water. |
| 3. | Granulate the mass of step 1 with solution of step 2. |
| 4. | Pass the wet mass through sieve of mesh size 20 and dry. |
| 5. | Pass the dried granule through sieve of mesh size 30. |

B. Coating of the Granules in FBC (Fluid Bed Coater)

% w/w

Ingredients

| | | |
|---|---|---|
| i) | Ethyl cellulose (Surelease ®) | 12.50 |
| | (Dry polymer weight of 25% w/w dispersion) | |
| ii) | Polycarbophil | 0.18 |
| iii) | Talc | 6.25 |
| iv) | Triethyl Citrate | 2.50 |
| v) | Colour Lake of Ponceau 4R | 0.10 |
| vi) | Water | Lost in processing |

Procedure:

| | |
|---|---|
| 1. | Mix (ii), (iii) and (v). |
| 2. | Pass mass of step 1 through sieve of mesh no. 100. |
| 3. | Disperse the bulk of step 2 in (vi) and pass through a Colloid mill. |
| 4. | Add (i) and (iv) to the bulk of step 3 and stir. |
| 5. | Coat the granules of part A in FBC with solution of step 4. |

C. Compression

mg/tablet

Ingredient

| | | |
|---|---|---|
| i) | Amoxicillin granules (coated in B) | 1361.14 |
| ii) | Microcrystalline cellulose | 100.00 |
| iii) | Croscarmellose sodium | 50.00 |
| iv) | Talc | 10.00 |
| v) | Magnesium stearate | 10.00 |

Procedure:

| | |
|---|---|
| 1. | Mix (ii), (iii), (iv) and (v) |
| 2. | Pass the mixture of step 1 through mesh no. 40 and blend with (i) |
| 3. | Compress the blended granules into tablets. |

### Example 12

A. Core Granules

mg/tablet

Ingredients

| | | |
|---|---|---|
| i) | Amoxicillin trihydrate | 860.00 |
| | (equivalent to 750 mg of Amoxicillin) | |
| ii) | Eudragit L-100 | 100.00 |
| iii) | Polycarbophil | 40.00 |
| iv) | Eudragit L100 | 20.00 |
| v) | Ethanol | Lost in processing |
| vi) | Purified Water | Lost in processing |

Procedure:

| | |
|---|---|
| 1. | Mix (i), (ii) and (iii) pass through mesh size 30. |
| 2. | Dissolve (iv) in a mixture of (v) and (vi) (6:4 ratio) |
| 3. | Granulate the mass of step 1 with solution of step 2. |
| 4. | Pass the wet mass through sieve of mesh size 20 and dry. |
| 5. | Pass the dried granule through sieve of mesh size 30. |

B. Coating of the Granules in FBC (Fluid Bed Coater)

% w/w

Ingredients

| | | |
|---|---|---|
| i) | Eudragit L-100 | 12.50 |
| ii) | Polycarbophil | 0.625 |
| iii) | Triethyl Citrate | 2.50 |
| iv) | Isopropyl Alcohol | Lost in processing |
| v) | Dichloromethane | Lost in processing |
| vi) | Colour Lake of Ponceau 4R | 0.10 |

Procedure:

| | |
|---|---|
| 1. | Mix (i) and (ii) and pass through mesh no. 100. |
| 2. | Pass (vi) through sieve of mesh no. 120. |
| 3. | Disperse the bulk of step 1 and 2 in 1:2 mixture of (iv) and (v) |
| 4. | Add (iii) to the bulk of step 3 and stir. |
| 5. | Coat the granules of part A in FBC with solution of step 4. |

C. Compression

mg/tablet

Ingredient

| | | |
|---|---|---|
| i) | Amoxicillin granules (coated in B) | 1180.39 |
| ii) | Microcrystalline cellulose | 100.00 |
| iii) | Croscarmellose sodium | 50.00 |
| iv) | Talc | 10.00 |
| v) | Magnesium stearate | 10.00 |

Procedure:

| | |
|---|---|
| 1. | Mix (ii), (iii), (iv) and (v) |
| 2. | Pass the mixture of step 1 through mesh no. 40 and blend |
| | with (i) |
| 3. | Compress the blended granules into tablets. |

### Example 13

A. Core Granules

mg/tablet

Ingredients

| | | |
|---|---|---|
| i) | Amoxicillin trihydrate | 860.00 |
| | (equivalent to 750 mg of Amoxicillin) | |
| ii) | Eudragit L-100 | 100.00 |
| iii) | Polycarbophil | 40.00 |
| iv) | Eudragit L100 | 20.00 |
| v) | Ethanol | Lost in processing |
| vi) | Purified Water | Lost in processing |

Procedure:

| | |
|---|---|
| 1. | Mix (i), (ii) and (iii) pass through mesh size 30. |
| 2. | Dissolve (iv) in a mixture of (v) and (vi) (6:4 ratio) |
| 3. | Granulate the mass of step 1 with solution of step 2. |
| 4. | Pass the wet mass through sieve of mesh size 20 and dry. |
| 5. | Pass the dried granule through sieve of mesh size 30. |

B. Coating of the Granules in FBC (Fluid Bed Coater)

% w/w

Ingredients

| | | |
|---|---|---|
| i) | Eudragit L-100 | 12.50 |
| ii) | Clavulanate Potassium | 12.25 |
| iii) | Polycarbophil | 0.625 |
| iv) | Triethyl Citrate | 2.50 |
| v) | Isopropyl Alcohol | Lost in processing |
| vi) | Dichloromethane | Lost in processing |
| vii) | Colour Lake of Ponceau 4R | 0.10 |

Procedure:

| | |
|---|---|
| 1. | Mix (i), (ii) and (iii). |
| 2. | Pass (vii) through sieve of mesh no. 120. |
| 3. | Disperse the bulk of step 1 and 2 in 1:2 mixture of (v) and (vi) |
| 4. | Add (iv) to the bulk of step 3 and stir. |
| 5. | Coat the granules of part A in FBC with solution of step 4. |

C. Compression

mg/tablet

Ingredient

| | | |
|---|---|---|
| i) | Amoxicillin granules (coated in B) | 1305.34 |
| ii) | Microcrystalline cellulose | 100.00 |
| iii) | Croscarmellose sodium | 50.00 |
| iv) | Talc | 10.00 |
| v) | Magnesium stearate | 10.00 |

Procedure:

| | |
|---|---|
| 1. | Mix (ii), (iii), (iv) and (v) |
| 2. | Pass the mixture of step 1 through mesh no. 40 and blend |
| | with (i) |
| 3. | Compress the blended granules into tablets. |

### Example 14

A. Core Granules

mg/tablet

Ingredients

| | | |
|---|---|---|
| i) | Amoxicillin trihydrate | 860.00 |
| | (equivalent to 750 mg of Amoxicillin) | |
| ii) | Eudragit L-100 | 100.00 |
| iii) | Polycarbophil | 40.00 |
| iv) | Eudragit L100 | 20.00 |
| v) | Ethanol | Lost in processing |
| vi) | Purified Water | Lost in processing |

Procedure:

| | |
|---|---|
| 1. | Mix (i), (ii) and (iii) pass through mesh size 30. |
| 2. | Dissolve (iv) in a mixture of (v) and (vi) (6:4 ratio) |
| 3. | Granulate the mass of step 1 with solution of step 2. |
| 4. | Pass the wet mass through sieve of mesh size 20 and dry. |
| 5. | Pass the dried granule through sieve of mesh size 30. |

B. Coating of the Granules in FBC (Fluid Bed Coater)

% w/w

Ingredients

| | | |
|---|---|---|
| i) | Eudragit L-100 | 12.50 |
| ii) | Polycarbophil | 0.625 |
| iii) | Triethyl Citrate | 2.50 |
| iv) | Isopropyl Alcohol | Lost in processing |
| v) | Dichloromethane | Lost in processing |
| vi) | Colour Lake of Ponceau 4R | 0.10 |

Procedure:

| | |
|---|---|
| 1. | Mix (i) and (ii) and pass through mesh no. 100. |
| 2. | Pass (vi) through sieve of mesh no. 120. |
| 3. | Disperse the bulk of step 1 and 2 in 1:2 mixture of (iv) and (v) |
| 4. | Add (iii) to the bulk of step 3 and stir. |
| 5. | Coat the granules of part A in FBC with solution of step 4. |

C. Preparation of Amoxicillin SR Granules

mg/tablet

Ingredient

| | | |
|---|---|---|
| i) | Amoxicillin granules (coated in B) | 1180.39 |
| ii) | Microcrystalline cellulose | 100.00 |
| iii) | Croscarmellose sodium | 50.00 |
| iv) | Talc | 10.00 |
| v) | Magnesium stearate | 10.00 |

Procedure:

| | |
|---|---|
| 1. | Mix (ii), (iii), (iv) and (v) |
| 2. | Pass the mixture of step 1 through mesh no. 40 and blend |
| | with (i) |

D. Preparation of Claculanate Potassium Granules

mg/tablet

Ingredient

| | | |
|---|---|---|
| i) | Clavulanate Potassium/ | 250.00 |
| | Microcrystalline Cellulose 1:1 mixture | |
| | (equivalent to 125 mg Clavulanic acid) | |
| ii) | Croscarmellose sodium | 50.00 |
| iii) | Talc | 10.00 |
| iv) | Magnesium stearate | 10.00 |

Procedure:

| | |
|---|---|
| 1. | Mix (i), (ii), (iii) and (iv) |
| 2. | Slug and de-slug the blend of step 1 and pass through sieve |
| | of mesh size 30. |

E. Compression into Inlay Tablets

Compress the granules of Amoxicillin SR granules and Clavulanate potassium granules into inlay tablets where the Clavulanate potassium granules are inlayed into the tablet of amoxicillin granules.

1-32. (canceled)

33. A rapidly disintegrating oral controlled release pharmaceutical composition comprising at least one active ingredient, and a polymer system comprising of at least two polymers wherein one is an acid insoluble polymer and the other is a bioadhesive polymer, which retard the release of the active ingredient in the stomach while providing rapid release of the active ingredient in the pH above 5.5, optionally with other pharmaceutically acceptable excipients.

34. The composition according to claim 33, wherein said active ingredient is selected from the group comprising antibiotics, such as cephalospoins and penicillins, and their pharmaceutically acceptable salts, hydrates, polymorphs, esters, and derivatives thereof.

35. The composition according to claim 33, wherein said active ingredient is amoxicillin trihydrate.

36. The composition according to claim 33, which comprises at least two active ingredients selected from the group consisting of amoxicillin, ampicillin, cloxacillin, clavulanic acid and cephalosporins, or pharmaceutically acceptable salts or derivatives thereof.

37. The composition according to claim 33, wherein the polymer system comprises polymers selected from the group consisting of polyvinyl pyrrolidone, polyvinyl acetate, methacrylic acid polymers, acrylic acid polymers, hydroxypropyl methylcellulose phthalate, hydroxypropyl methylcellulose acetate succinate, cellulose acetate phthalate, cellulose acetate butyrate, cellulose acetate propionate, alginates, cellulose derivative, polyethylene oxide, chitosans, and polycarbophil, or a mixture thereof.

38. The composition according to claim 33, wherein the acid insoluble polymer is selected from the group consisting of methacrylic acid polymers, acrylic acid polymers, hydroxypropyl methylcellulose phthalate, hydroxypropyl methylcellulose acetate succinate, cellulose acetate phthalate, cellulose acetate butyrate, cellulose acetate propionate, and alginates, or a mixture thereof.

39. The composition according to claim 33, wherein the bioadhesive polymer is selected from the group consisting of polycarbophil and chitosans.

40. The composition according to claim 37, wherein the polymer system comprises methacrylic acid polymer and polycarbophil.

41. The composition according to claim 33, which additionally comprises a cellulose derivative.

42. The composition according to claim 34, which additionally comprises a cellulose derivative.

43. The composition according to claim 35, which additionally comprises a cellulose derivative.

44. The composition according to claim 36, which additionally comprises a cellulose derivative.

45. The composition according to claim 39, which additionally comprises a cellulose derivative.

46. The composition according to claim 41, wherein the cellulose derivative is selected from the group consisting of alkyl cellulose and carboxyalkyl cellulose.

47. The composition according to claim 40, wherein the ratio of methacrylic acid polymer and polycarbophil is 10:1 to 1:10 by weight of the composition.

48. A process for preparation of a composition according to claim 33, which comprises the step of:
i) mixing of active ingredient(s) and polymer(s),
ii) optionally adding one or more other pharmaceutically acceptable excipients, and
iii) formulation of the mixture into a suitable dosage form.

49. The process according to claim 48, wherein said active ingredient is amoxicillin trihydrate.

50. The process according to claim 16, which comprises at least two active ingredients selected from the group consisting of amoxicillin, ampicillin, cloxacillin, clavulonic acid, and cephalosporins, or pharmaceutically acceptable salts or derivatives thereof.

51. The process according to claim 48, wherein the composition additionally comprises a cellulose derivative.

52. The process according to claims 49, wherein the composition additionally comprises a cellulose derivative
